(19) United States
(12) Patent Application Publication
(10) Pub. No.: US 2025/0295862 A1
(43) Pub. Date: Sep. 25, 2025

(54) MECHANICALLY ACTUATED NEEDLE SHIELDING DEVICE FOR METERED DOSE SYRINGES

(71) Applicant: RAMU VUPPALA, Midland, TX (US)

(72) Inventor: RAMU VUPPALA, Midland, TX (US)

(21) Appl. No.: 19/231,723

(22) Filed: Jun. 9, 2025

(51) Int. Cl. A61M 5/32

(52) U.S. Cl. CPC A61M 5/3202

(57) ABSTRACT

Exemplary embodiments of the present disclosure are directed towards a mechanically actuated needle shielding device for metered dose syringes. The device includes a hub cap configured to mount over a needle hub without requiring modification to the syringe, and a shield slide positioned coaxially with respect to the hub cap. The shield slide is movable along a longitudinal axis between a retracted position, in which the needle is exposed for injection, and an extended position, in which the needle is concealed. A shield slide magnet and a hub cap magnet are embedded in respective components with like poles facing each other, generating a magnetic repelling force that biases the shield slide toward the extended position. During injection, the shield slide retracts under axial force and automatically returns to the extended position upon release, thereby concealing the needle post-use to enhance safety and minimize accidental exposure.

### COPYRIGHT AND TRADEMARK NOTICE

This application includes material which is subject or may be subject to copyright and/or trademark protection. The copyright and trademark owner(s) have no objection to the facsimile reproduction by any of the patent disclosure, as it appears in the Patent and Trademark Office files or records, but otherwise reserves all copyright and trademark rights whatsoever.

### TECHNICAL FIELD

The present invention relates generally to the field of medical injection devices, and more particularly to a mechanically actuated needle shielding device for metered dose syringes that enhances safety and reduces psychological trauma during and after injection, especially in pediatric applications.

### BACKGROUND

Medical syringes are essential tools used in the delivery of medications, vaccines, and other therapeutic agents through parenteral administration. Among them, metered dose syringes play a critical role in accurately dispensing specific volumes of medication-especially in cases where the therapeutic agents are potent, costly, or require precise dosing regimens. These syringes are widely adopted across clinical, pediatric, and home-care settings.

A common application of metered dose syringes is in pediatric treatments where recurring doses are administered over prolonged durations. For example, children undergoing hormone therapy or chronic vaccination schedules may require frequent injections. Although clinically necessary, the repeated visual exposure to a hypodermic needle can lead to emotional distress, anticipatory anxiety, and injection phobia in pediatric patients. This psychological barrier often leads to increased resistance, fear-based responses, and diminished treatment compliance.

Current syringe designs, while effective in delivering precise medication volumes, typically expose the needle prior to and during administration. This visual cue can intensify the patient's fear, especially in children, potentially affecting the overall injection experience. Furthermore, existing needle shielding solutions, where available, often require structural modifications to the syringe body, are incompatible across various needle hub formats, or fail to consistently ensure user safety after the injection is complete. Some mechanisms lack smooth retraction or automatic shielding features, increasing the risk of accidental needle sticks and complicating disposal.

There is, therefore, a clear need for an improved syringe accessory or integrated mechanism that not only delivers precise medication volumes but also conceals the needle effectively during non-injection phases, reduces psychological trauma for patients—especially children—and promotes safer handling for healthcare providers. Such a solution must be mechanically simple, compatible with standard syringe and needle hub configurations, and must operate seamlessly during the injection cycle—ideally transitioning between exposed and concealed states without complex user interaction.

In light of the existing problems and limitations, the present invention provides a mechanically actuated needle shielding device specifically designed for metered dose syringes, offering enhanced safety, ease of use, and trauma-reducing functionality.

### SUMMARY

The following invention presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

An objective of the present disclosure is directed towards providing a needle shielding device for metered dose syringes that reduces visual exposure to the needle, thereby minimizing fear and anxiety in patients, especially children.

An objective of the present disclosure is directed towards enabling a mechanically actuated shielding mechanism that transitions between shielded and unshielded states in a smooth and controlled manner without requiring complex user interaction.

An objective of the present disclosure is directed towards ensuring compatibility of the needle shielding device with conventional syringe barrels and standard needle hubs, without requiring structural modifications to the existing syringe components.

An objective of the present disclosure is directed towards improving the safety of healthcare providers and caregivers by concealing the needle automatically after use, thereby reducing the risk of accidental needle-stick injuries.

An objective of the present disclosure is directed towards offering a compact and reliable device that integrates with metered dose syringes without hindering the accuracy or consistency of fluid delivery.

An objective of the present disclosure is directed towards providing a needle shielding device that can be easily assembled, disassembled, or replaced, allowing for convenient cleaning, sterilization, or disposal.

An objective of the present disclosure is directed towards delivering a child-friendly injection experience by preventing the visual stimulus of the needle, which often contributes to psychological trauma and treatment non-compliance.

An objective of the present disclosure is directed towards offering a mechanically simple solution that does not rely on electronic components, ensuring robust and cost-effective manufacturing and usage.

In an exemplary embodiment of the present disclosure, the needle shielding device comprises a shield slide that is configured to move linearly along the syringe barrel. This slide is positioned to cover or uncover the hypodermic needle depending on the state of syringe operation. When the syringe is not in use, the shield remains in a forward position, thereby concealing the needle from view and reducing visual anxiety for patients, particularly children. During administration, the shield retracts to allow exposure of the needle, thus enabling smooth drug delivery.

Another exemplary embodiment of the present disclosure, a pair of opposing magnets—namely the shield slide magnet and the hub cap magnet—are strategically embedded within the shield components to provide magnetically actuated control over the shield's movement. These magnetic elements generate repulsive or attractive forces at specific stages of operation, enabling passive extension and retraction of the shield without the need for user intervention. This mechanical simplicity enhances usability and safety by reducing manual contact with the needle.

Another exemplary embodiment of the present disclosure, a pair of opposing magnets—comprising a shield slide magnet and a hub cap magnet—are strategically embedded within the needle shielding components to enable magnetically actuated control over the movement of the shield slide. These magnets may be oriented with like poles facing one another, thereby generating repulsive forces that store potential energy when compressed and exert a restoring force when released. This magnetic interaction may facilitate passive retraction and automatic extension of the shield slide along the longitudinal axis in response to external axial force and subsequent release. The use of magnetically derived actuation may eliminate the need for traditional mechanical springs, thereby simplifying the design while enhancing operational reliability, minimizing user intervention, and improving overall safety during and after injection procedures.

Another exemplary embodiment of the present disclosure, the hub cap and needle hub are designed for secure engagement, allowing the shielding device to integrate with standard syringe formats. This ensures compatibility with commonly used needle assemblies, thereby eliminating the need for syringe redesign or proprietary connections. The system supports interchangeable use across brands and models, fulfilling the objective of broad applicability and ease of adoption.

Another exemplary embodiment of the present disclosure, the plunger and barrel of the syringe remain functionally unaffected by the addition of the shielding device. The shielding mechanism operates independently from the dose metering and fluid delivery functions, ensuring that the accuracy of medication volume is preserved. This design maintains the syringe's core functionality while enhancing safety and comfort.

Another exemplary embodiment of the present disclosure, the device incorporates a flange and thumb rest at the rear end of the plunger to assist the operator in stabilizing the syringe during injection. This ergonomic enhancement contributes to safer and more controlled administration, especially in pediatric or home-use contexts.

Another exemplary embodiment of the present disclosure, the device is designed with modular construction such that the shielding components can be disassembled for cleaning or disposal after use. The magnetically coupled parts can be easily separated and reassembled, facilitating hygiene maintenance and reusability or safe discarding, depending on the application requirements.

### BRIEF DESCRIPTION OF THE DRAWINGS

In the following, numerous specific details are set forth to provide a thorough description of various embodiments. Certain embodiments may be practiced without these specific details or with some variations in detail. In some instances, certain features are described in less detail so as not to obscure other aspects. The level of detail associated with each of the elements or features should not be construed to qualify the novelty or importance of one feature over the others.

FIG. 1 is an isometric view of the syringe equipped with the needle shielding device shown in a pre-engaged configuration, wherein the shield is in a retracted position and the needle is exposed in a ready-to-use state.

FIG. 2 is a side view of the syringe equipped with the needle shielding device shown in an engaged configuration, wherein the shield remains in a retracted position and the needle is exposed for administration.

FIG. 3 is a sectional view of the syringe equipped with the needle shielding device, taken along section 3-3 as shown in FIG. 2, illustrating the internal arrangement of components in the engaged configuration.

FIG. 4 is an enlarged sectional view of the syringe equipped with the needle shielding device, illustrating the detailed portion of FIG. 3, wherein the needle shielding device is shown attached to the needle hub in the engaged configuration.

FIG. 5 is an isometric view of the syringe equipped with the needle shielding device shown in a shielded configuration, wherein the shield is in an extended position covering the needle after administration.

FIG. 6 is a side view of the syringe equipped with the needle shielding device shown in a shielded configuration, wherein the shield remains in an extended position and the needle is concealed.

FIG. 7 is a sectional view of the syringe equipped with the needle shielding device, taken along section 7-7 as shown in FIG. 6, illustrating the internal arrangement of components in the shielded configuration.

FIG. 8 is an enlarged sectional view of the syringe equipped with the needle shielding device, illustrating the detailed portion of FIG. 7, wherein the needle shielding device is shown attached to the needle hub in the shielded configuration.

FIG. 9 is an isometric view of the syringe equipped with the needle shielding device shown in an exploded configuration, illustrating the individual components of the device in a disassembled state.

FIG. 10 is a side view of the syringe equipped with the needle shielding device shown in an exploded configuration, illustrating the linear arrangement of all internal and external components along the longitudinal axis of the syringe.

FIG. 11 is a sectional view of the syringe equipped with the needle shielding device, taken along section 11-11 as shown in FIG. 10, illustrating the internal construction and sequential assembly of the components along the longitudinal axis.

### DETAILED DESCRIPTION OF EXAMPLE EMBODIMENTS

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and so forth, herein do not denote any order, quantity, or importance, but rather are used to distinguish one element from another.

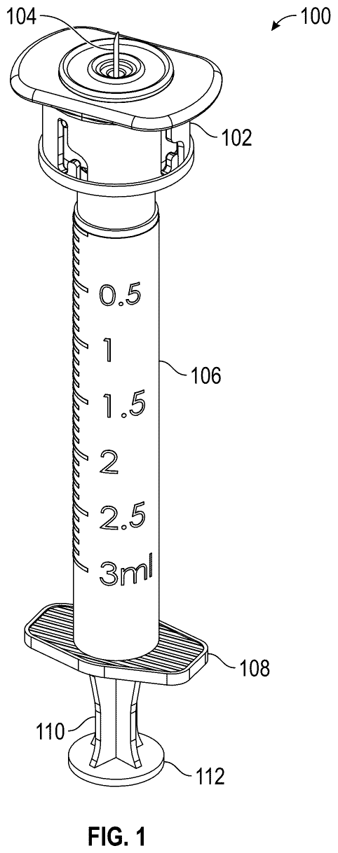

Referring to FIG. 1, it is an isometric view of the syringe equipped with the needle shielding device shown in a pre-engaged configuration, wherein the shield is in a retracted position and the needle is exposed in a ready-to-use state. The overall syringe assembly is generally indicated by reference numeral 100. The device includes a needle shielding device 102, which may be mechanically integrated or removably attached near the front end of the syringe. In this configuration, the shielding component may remain in a passive or standby state, permitting unobstructed visibility and access to the needle 104, which may be used for intramuscular, subcutaneous, or other forms of injection depending on the medical application.

The syringe body comprises a barrel 106, which may be calibrated for accurate measurement of fluid medication. The barrel is ergonomically supported by a flange 108, which may aid the user in stabilizing the syringe during injection. A plunger 110 is slidably positioned within the barrel and may be actuated to expel the contents of the syringe through the needle. At the rear end of the plunger, a thumb rest 112 is provided to facilitate controlled and comfortable operation during manual depression. In this figure, all primary elements of the syringe are visible and aligned in a configuration suggesting that the device is prepped and ready for use, with the shielding mechanism held in a retracted state for clear access to the needle.

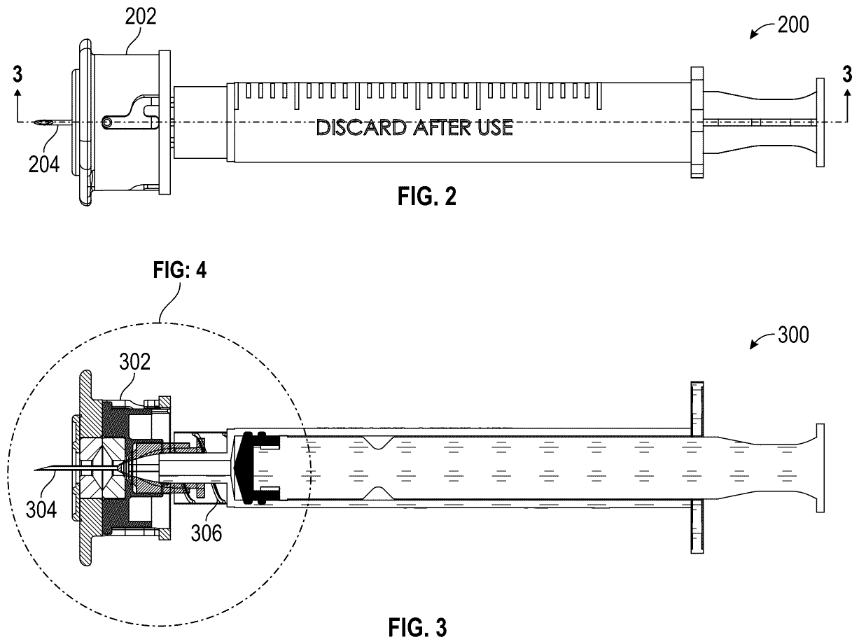

Referring to FIG. 2, it is a side view of the syringe equipped with the needle shielding device shown in an engaged configuration, wherein the shield remains in a retracted position and the needle is exposed for administration. The overall syringe assembly is indicated by reference numeral 200. In this view, the needle shielding device 202 may be mechanically coupled to the front end of the syringe, aligned concentrically with the barrel and designed to allow unimpeded exposure of the needle 204 during injection. The configuration illustrated here may correspond to the state of the syringe at the moment of administration, where the shielding mechanism remains retracted, enabling the needle to be inserted into the target tissue.

The side profile may provide a clearer understanding of the linear arrangement of the device components and their functional orientation during use. The needle shielding device may still maintain readiness to return to a shielded state post-injection, but during this stage, it may ensure that the shielding does not interfere with the accuracy or depth of the injection. This view may also be helpful in appreciating the spatial relationship between the needle and the surrounding components of the syringe, supporting a better understanding of the device's functional operation during medication delivery.

Referring to FIG. 3, it is a sectional view of the syringe equipped with the needle shielding device, taken along section 3-3 as shown in FIG. 2, illustrating the internal arrangement of components in the engaged configuration. The overall structure is indicated by reference numeral 300. In this view, the needle shielding device 302 is shown in a retracted position, wherein it may be designed to allow the needle 304 to protrude from the assembly, making it available for administration of medication. The sectional cut enables a clear visualization of the interaction between the internal shielding components and the core drug delivery path.

An important feature illustrated in this view is the spring 306, which may optionally be positioned within the needle shielding device to facilitate controlled movement of the shield slide during operation. In certain exemplary embodiments, the spring may serve to bias the shield slide toward its extended position under default conditions and compress or retract in response to external axial force during injection, thereby allowing the needle to be exposed. Alternatively, or in combination, magnetic repulsion between a pair of like-poled magnets embedded in the shield slide and hub cap may provide a similar biasing effect without reliance on mechanical deformation. This configuration may ensure that needle exposure occurs only during deliberate actuation, thereby enhancing safety and user control. The sectional view may also demonstrate the coaxial alignment of the shielding elements with the syringe barrel and needle assembly, contributing to the structural stability and responsiveness of the device.

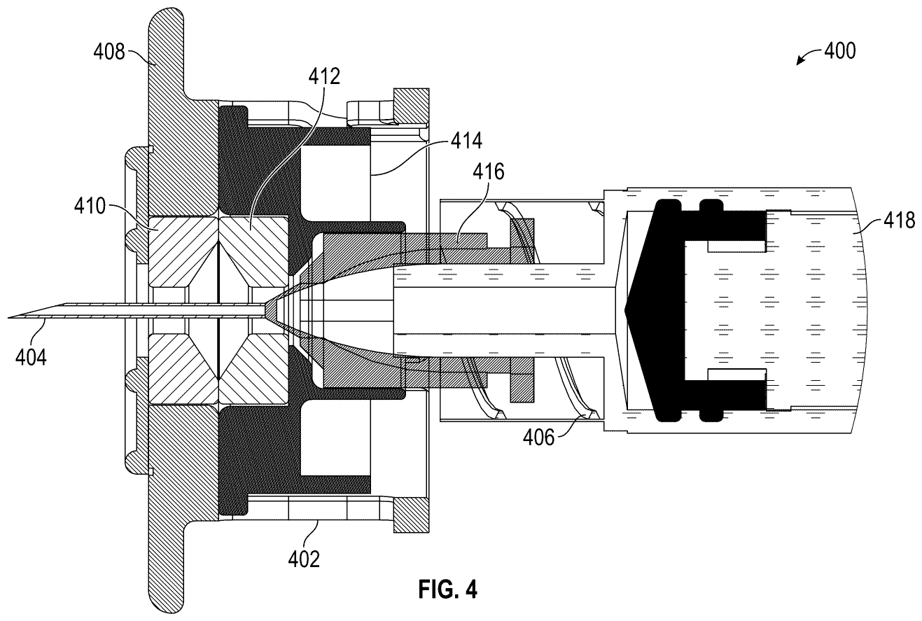

Referring to FIG. 4, it is an enlarged sectional view of the syringe equipped with the needle shielding device, illustrating the detailed portion of FIG. 3, wherein the needle shielding device is shown attached to the needle hub in the engaged configuration. The overall assembly is designated by reference numeral 400. This view may provide a magnified understanding of the mechanical interplay between the internal components during the injection phase.

The needle shielding device 402 may be depicted in its retracted state, wherein the needle 404 protrudes forward to enable fluid administration. The shield slide 408, shown in this position, is a movable component aligned along the longitudinal axis and configured to extend forward to enclose the needle when not in use. A spring 406 may optionally be positioned internally between the shield slide and the hub cap and may serve to bias the shield slide toward the extended position. In certain exemplary embodiments, magnetic repulsion between a shield slide magnet and a hub cap magnet may be solely responsible for driving the shield slide forward after the injection force is released. This dual-capability approach allows the shielding device to automatically return to a concealed configuration, thereby reducing visual exposure of the needle and minimizing the risk of accidental contact.

Adjacent to the shield slide, a shield slide magnet 410 is embedded in a position aligned along the longitudinal axis to interact magnetically with a corresponding hub cap magnet 412 mounted within the hub cap 414. These magnets may be oriented such that like poles face each other, thereby generating a repulsive force that urges the shield slide forward into the extended, needle-concealing position. When an external axial force is applied during injection, this repulsive force is temporarily overcome, allowing the shield slide to retract. Upon release of the force, the repelling magnetic interaction may cause the shield slide to return to its extended position. This configuration may eliminate the need for manual shielding actions, enabling automatic or semi-automatic actuation through magnetic force alone.

The needle hub 416 provides structural support for securing the needle assembly and facilitating its alignment with the shielding device. Fluid within the syringe may be expelled via the plunger 418, which, when pressed, allows the medication to be delivered through the needle during the engaged state. The arrangement of all these components in this figure may highlight the integrated and coordinated functionality of the needle shielding mechanism with standard syringe operation.

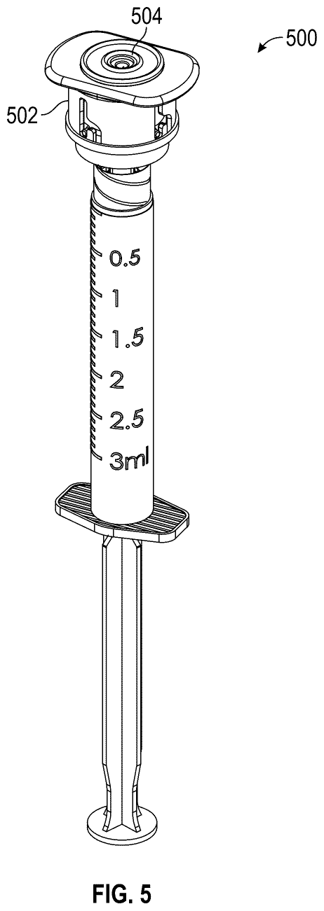

Referring to FIG. 5, it is an isometric view of the syringe equipped with the needle shielding device shown in a shielded configuration, wherein the shield is in an extended position covering the needle after administration. The overall assembly is designated by reference numeral 500. This view may represent the post-injection state of the syringe, where the shielding mechanism has moved forward to enclose the needle for enhanced safety and protection.

The needle shielding device 502 may be positioned concentrically with respect to the barrel and front end of the syringe, forming an external protective barrier. The shielding component may be actuated primarily through magnetic repulsion generated by like-pole alignment of embedded magnets within the shield slide and the hub cap, enabling the shield to advance forward into a concealing position. In some exemplary embodiments, a spring may optionally be included to supplement or reinforce the extension movement. In the illustrated configuration, the shield has moved to the forward position, concealing the needle 504, thereby minimizing the risk of accidental needle-stick injuries and shielding the needle from view to reduce psychological discomfort, particularly in pediatric or needle-sensitive users.

This shielded configuration may also facilitate safer handling and disposal of the syringe after use, aligning with healthcare safety protocols. The isometric view may aid in understanding the spatial relationship of all external components and the protective coverage achieved by the shielding device.

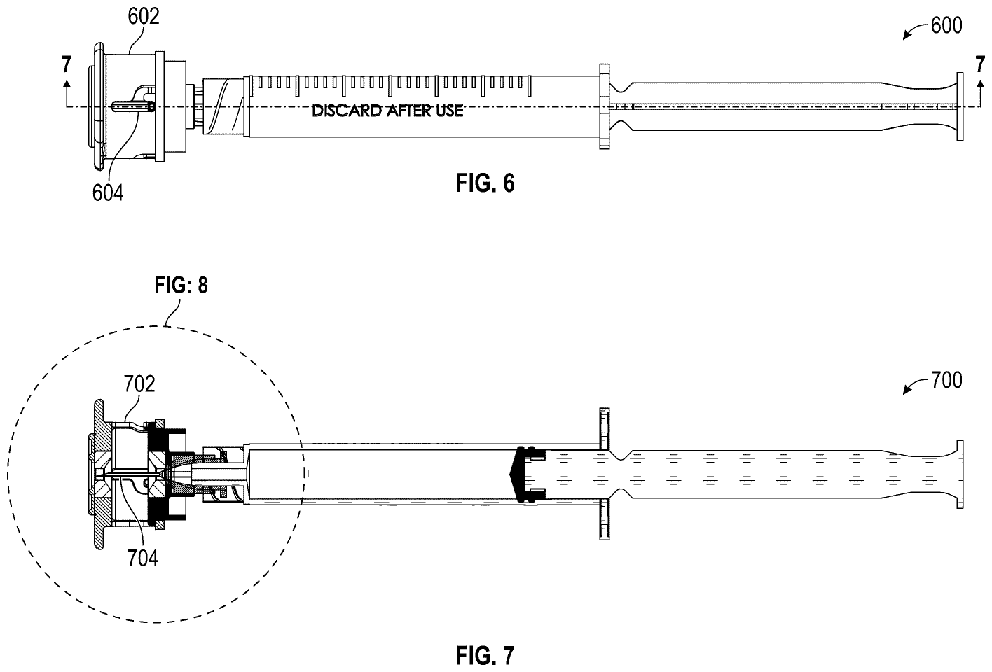

Referring to FIG. 6, it is a side view of the syringe equipped with the needle shielding device shown in a shielded configuration, wherein the shield remains in an extended position and the needle is concealed. The overall syringe assembly is indicated by reference numeral 600. This view may represent the state of the device following injection or during transport, where user safety and needle concealment are prioritized.

The needle shielding device 602 may be designed to transition from a retracted to an extended position primarily through magnetic repulsion between like poles of embedded magnets within the shield slide and the hub cap. In this shielded configuration, the shielding device may advance forward to envelop the distal end of the syringe, thereby concealing the needle 604 and ensuring it is no longer visible or accessible. In certain exemplary embodiments, this magnetic actuation may be supplemented by a mechanical biasing element, such as a spring, to support consistent forward movement and positioning of the shield.

This arrangement may serve to protect users from accidental contact with the used needle, facilitate safer disposal, and reduce visual anxiety in patients who are sensitive to needles. The side view offers a linear perspective of the syringe assembly and may help demonstrate how the shield aligns with and surrounds the needle upon completion of the injection cycle.

Referring to FIG. 7, it is a sectional view of the syringe equipped with the needle shielding device, taken along section 7-7 as shown in FIG. 6, illustrating the internal arrangement of components in the shielded configuration. The overall assembly is designated by reference numeral 700. This view may provide a clearer understanding of how the internal components of the shielding mechanism are positioned relative to the syringe barrel and plunger after the injection has been completed.

The needle shielding device 702 may be configured to advance forward primarily under magnetic repulsion between opposing magnets embedded within the shield slide and the hub cap, thereby enclosing the needle 704. In this shielded configuration, the needle is concealed, and the shield may function as a protective barrier to reduce the risk of accidental contact or exposure. In some non-limiting exemplary embodiments, the movement may be further assisted by a biasing element such as a spring. The sectional view may illustrate how the movable shield nests over the distal end of the syringe and maintains coaxial alignment along the longitudinal axis of the barrel, ensuring reliable operation during and after injection.

This configuration may not only improve operational safety post-injection but also facilitate improved handling, transport, or disposal by healthcare workers or caregivers. The view highlights how the shielding mechanism remains functionally integrated without obstructing core injection operations.

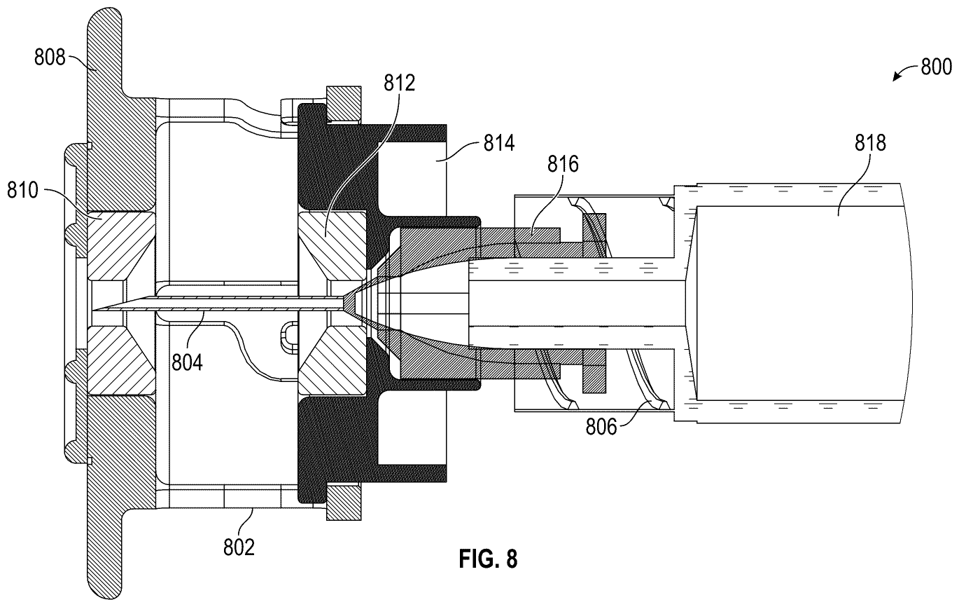

Referring to FIG. 8, it is an enlarged sectional view of the syringe equipped with the needle shielding device, illustrating the detailed portion of FIG. 7, wherein the needle shielding device is shown attached to the needle hub in the shielded configuration. The overall assembly is designated by reference numeral 800. This enlarged view may provide a comprehensive understanding of the structural interaction between the shielding mechanism and the injection components once the device has transitioned to its safety state.

The needle shielding device 802 may include a shield slide 808, which in the depicted configuration is in the forward or extended position, thereby fully enclosing the needle 804 in a concealed state. The movement of the shield slide may primarily be driven by magnetic repulsion between like-polarity magnets embedded within the shield slide and the hub cap. In some non-limiting exemplary embodiments, an optional spring 806 may be positioned to provide supplemental biasing force, thereby assisting the return of the shield slide to its extended position following injection. This combination of magnetic repulsion and optional spring assistance may contribute to effective needle concealment and enhanced user safety.

A shield slide magnet 810 is embedded within the shield slide and may be configured with like polarity to a corresponding hub cap magnet 812 housed within the hub cap 814. This arrangement of like poles may generate a repelling magnetic force that biases the shield slide toward the extended position, effectively functioning as a magnetic actuation mechanism. The magnetic interaction may provide both motion and positional stability to the shield slide, depending on the operational state of the syringe. In some non-limiting exemplary embodiments, an optional mechanical spring may be included to supplement the magnetic force, offering enhanced control and redundancy in shield actuation. This magnetically coordinated mechanism may reduce the need for complex mechanical linkages while preserving reliable shielding functionality.

The needle hub 816 provides structural alignment and fluid communication between the syringe and the needle assembly. Meanwhile, the plunger 818 may remain in a static or post-actuated position following the completion of the fluid delivery. This view may effectively demonstrate how the safety mechanism engages with the core syringe structure to securely shield the needle after use, thereby promoting safer handling and reducing risk to patients and caregivers.

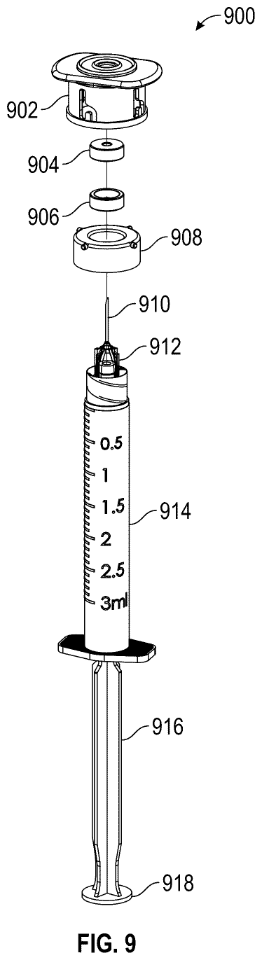

Referring to FIG. 9, it is an isometric view of the syringe equipped with the needle shielding device shown in an exploded configuration, illustrating the individual components of the device in a disassembled state. The overall layout is indicated by reference numeral 900. This view may provide a clear understanding of the modular construction of the syringe assembly and how the components are configured to interact during operation.

The shield slide 902 is shown separated from the main body and may be configured to move linearly along the longitudinal axis of the syringe between a retracted and an extended position. Embedded within the shield slide is a shield slide magnet 904, which may be oriented with like polarity relative to a corresponding hub cap magnet 906 housed within the hub cap 908. This like-pole configuration may generate a magnetic repelling force that biases the shield slide toward the extended position, functioning analogously to a magnetic spring. The hub cap 908 may serve both as a structural support and as a magnetic interface to guide and stabilize the axial movement of the shield. In some exemplary embodiments, an optional spring may also be incorporated between the shield slide and hub cap to supplement the repulsive magnetic force, offering additional control over the shield's return dynamics after injection.

The needle 910 and needle hub 912 are also illustrated, showing their alignment relative to the hub cap. The needle may be secured within the hub and aligned such that it extends forward through the shielding components during the engaged state, and remains enclosed during the shielded configuration. The barrel 914 forms the main cylindrical body of the syringe and may contain volume markings for metered dosing. A plunger 916 is configured to slide within the barrel for fluid delivery, while a thumb rest 918 is attached at the rear end of the plunger to allow comfortable and stable actuation during injection. This exploded view may assist in visualizing the positional relationships and assembly sequence of each component, highlighting the simplicity and integration of the needle shielding mechanism with the core syringe structure.

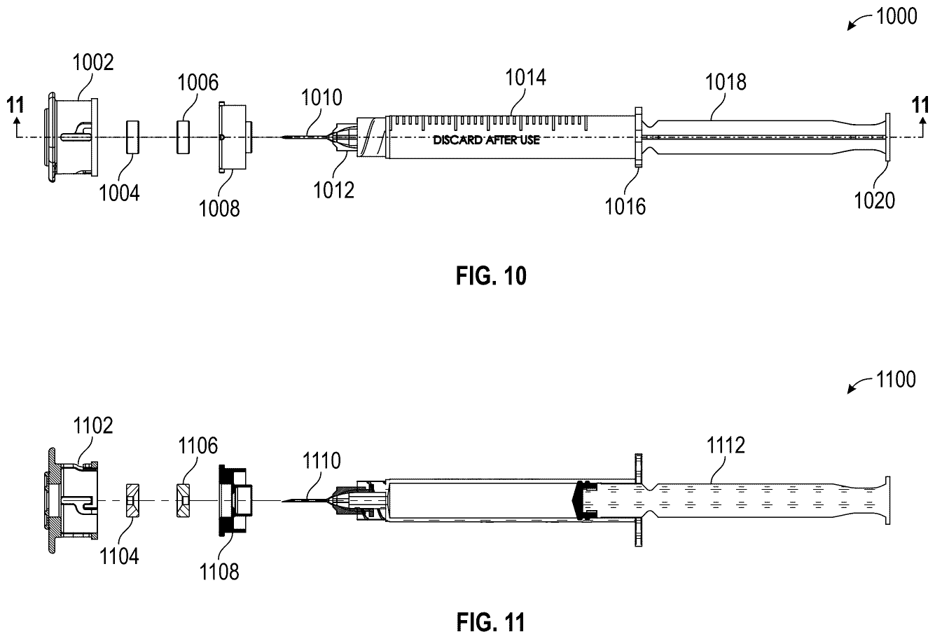

Referring to FIG. 10, it is a side view of the syringe equipped with the needle shielding device shown in an exploded configuration, illustrating the linear arrangement of all internal and external components along the longitudinal axis of the syringe. The overall assembly is designated by reference numeral 1000. This view may aid in understanding how each component aligns sequentially from the front (needle end) to the rear (plunger end), contributing to the assembly and functionality of the complete device, including the integration of a magnetically actuated shielding mechanism.

The shield slide 1002 is shown at the front of the assembly and may be configured to move along the longitudinal axis between an extended position to conceal the needle and a retracted position to expose it during injection. A shield slide magnet 1004 is embedded within the shield slide and may magnetically interact with a corresponding hub cap magnet 1006 positioned within the hub cap 1008. These magnets may be arranged with like poles facing each other to generate repulsive forces, thereby biasing the shield slide toward the extended position. In some exemplary embodiments, a spring may optionally be included between the shield slide and the hub cap to supplement the magnetic repulsion, offering additional restoring force and ensuring smooth actuation and reliable re-extension after injection.

The needle 1010 is secured by the needle hub 1012, which may be configured to maintain precise alignment and facilitate fluid communication with the barrel during administration. Moving proximally, the barrel 1014 defines the main fluid-retaining body of the syringe and is typically filled with the medication or vaccine. A flange 1016 is provided at the proximal end of the barrel to allow for stable grip and controlled operation by the user during injection.

Further along the axis, a plunger 1018 is positioned within the barrel and may be configured to slide axially to expel the fluid through the needle during injection. At the rear end of the plunger, a thumb rest 1020 is provided, which may offer ergonomic support to facilitate manual actuation. This exploded linear view may serve to illustrate the spatial orientation and dimensional compatibility of the various components, particularly highlighting how the shielding device integrates with conventional syringe architecture to deliver a safer and more user-friendly injection experience.

Referring to FIG. 11, it is a sectional view of the syringe equipped with the needle shielding device, taken along section 11-11 as shown in FIG. 10, illustrating the internal construction and sequential assembly of the components along the longitudinal axis. The overall system is indicated by reference numeral 1100. This view may provide an in-depth look at the internal alignment and integration of components, reinforcing how the shielding mechanism is designed to operate in conjunction with the syringe structure while maintaining axial symmetry and operational stability.

The shield slide 1102 is positioned near the distal end and may be configured to travel along the syringe's axis to either expose or enclose the needle during different phases of use. Embedded within the shield slide is the shield slide magnet 1104, which may magnetically interact with a corresponding hub cap magnet 1106 situated within the hub cap 1108. These magnets may be oriented with like poles facing one another to generate a repelling force that biases the shield slide toward the extended position. In certain exemplary embodiments, a spring may additionally be positioned between the shield slide and the hub cap to complement the magnetic repulsion and further support the return of the shield slide to its extended position following retraction. This magnetic and optional spring-assisted arrangement may enable controlled actuation and improved stability of the shield mechanism.

The needle 1110 is centrally aligned and extends forward through the shielding device, supported structurally by the hub cap. The configuration shown in this sectional view may reflect either an engaged or transitioning state, depending on the relative positions of the magnets and the shield slide. At the opposite end, the plunger 1112 is aligned within the barrel and may be used to displace fluid through the needle when depressed. The sectional layout may clearly demonstrate the coaxial arrangement of these critical elements, emphasizing how the shielding device functions in tandem with the syringe components to provide enhanced safety, user comfort, and ease of operation.

According to non-limiting exemplary aspects of the present invention, a mechanically actuated needle shielding device is provided for use with metered dose syringes. The shielding device comprises a hub cap configured to be mounted over the needle hub of a syringe, wherein the mounting may be achieved without requiring any modification to the syringe structure. The hub cap may engage the needle hub via a friction fit or threaded engagement, thereby allowing for compatibility with standard commercially available syringes.

Coaxially disposed with respect to the hub cap is a shield slide, which is configured to move along a longitudinal axis between a retracted position, in which a distal end of the needle is exposed for administration, and an extended position, in which the needle is concealed to prevent accidental contact and visual exposure. The shield slide may include a front opening aligned with the longitudinal axis of the needle, such that the needle can pass through the opening during injection and be enclosed when retracted.

The shield slide and hub cap each include embedded magnets-specifically, a shield slide magnet and a hub cap magnet-arranged such that like poles face one another. This configuration generates a magnetic repelling force along the longitudinal axis. When an external axial force is applied during injection, the shield slide retracts toward the hub cap against this repelling force. Upon release of the injection force, the magnetic repulsion automatically returns the shield slide to the extended position, thereby concealing the needle. This magnetic actuation mechanism performs the dual role of storing and releasing energy, functioning analogously to a spring, while offering a mechanically simple and reliable solution.

The device may be further configured such that in the extended position, the shield slide fully encloses the needle, thereby significantly reducing the risk of needle-stick injuries and minimizing visual exposure of the needle before and after administration. In certain aspects, the shield slide is configured to conceal the needle prior to, during, and after injection, which may be particularly advantageous in pediatric applications, where visual exposure to needles can cause emotional trauma or anxiety.

According to non-limiting exemplary aspects, the shielding device may include alignment features within the hub cap to ensure axial and rotational stability during actuation. The components are designed to operate without interfering with the syringe's core functionality, such as medication delivery through the plunger mechanism.

In one exemplary method, a method of shielding a needle of a metered dose syringe using the described device includes the steps of coupling the hub cap of the shielding device over the needle hub of the syringe, positioning the shield slide over the needle, and orienting the magnets such that like poles face each other to generate a magnetic repelling force. During injection, the shield slide retracts toward the hub cap under external axial force, and after injection, it automatically returns to the extended position under the influence of magnetic repulsion, thereby ensuring the needle is concealed post-use to enhance safety and reduce exposure.

In another exemplary method, a method of administering an injection to a pediatric patient includes preparing the syringe with a fluid medication and needle, attaching the needle shielding device comprising the hub cap, shield slide, and magnets to the syringe, and maintaining the shield slide in an extended position to conceal the needle prior to administration. Upon application of injection force, the shield slide retracts to expose the needle and deliver the medication. Following injection, the shield slide automatically returns to the extended position under magnetic force, thereby maintaining the needle in a concealed state both before and after injection to reduce injection-related anxiety and enhance safety.

According to non-limiting exemplary aspects of the present invention, while the primary mechanism for axial actuation of the shield slide relies on magnetic repulsion between like-oriented magnets embedded in the shield slide and hub cap, in certain alternative embodiments, a mechanical spring may additionally or optionally be positioned between the hub cap and the shield slide. The spring may be configured to bias the shield slide toward the extended position, thereby supplementing the repelling magnetic force. In such configurations, the combined action of the spring and magnetic repulsion may enhance the reliability and responsiveness of the shield slide's return motion following retraction. This embodiment may be particularly useful in use cases requiring greater actuation force or where redundant safety mechanisms are desired. However, it is understood that the invention may operate effectively using only magnetic repulsion without the inclusion of a spring.

According to non-limiting exemplary aspects of the present invention, the mechanically actuated needle shielding device may be particularly beneficial when used in pediatric medical environments, where frequent injections such as vaccinations, hormone therapies, or allergy treatments are administered. Children often exhibit psychological distress at the sight of needles, which can lead to injection refusal, anxiety, or long-term fear of medical procedures. By maintaining the shield slide in an extended position prior to injection, the device may prevent visual exposure of the needle, thereby reducing anticipatory fear. During the injection process, the shield slide retracts temporarily and automatically returns, ensuring that the needle is never unnecessarily exposed before or after use. This functionality may contribute to a trauma-reduced experience, making the device especially suitable for child-focused clinics, pediatric hospitals, and home-care environments.

According to non-limiting exemplary aspects of the present invention, the shielding device may also be employed in mass immunization programs, where rapid, repetitive administration of vaccines must be performed by healthcare workers. In such high-throughput environments, the risk of accidental needle-stick injuries increases significantly. The present shielding device, by automatically enclosing the needle after injection, enhances post-use safety, allowing practitioners to focus on administration without needing manual shielding or disposal actions. The spring-and-magnet-assisted return mechanism provides a passive and consistent method of needle concealment, thereby reducing fatigue, enhancing workflow efficiency, and supporting compliance with workplace safety protocols.

According to non-limiting exemplary aspects of the present invention, the shielding device may be utilized in home-injection therapy settings, where patients or caregivers self-administer medications for chronic conditions such as diabetes, growth hormone deficiencies, or autoimmune disorders. In such cases, users may lack professional training, and ensuring both ease of use and safe post-injection handling is essential. The shielding device allows for straightforward engagement with existing syringes and provides an intuitive visual cue that the injection is complete and the needle is safely concealed. This minimizes the risk of accidental reuse or improper disposal, making the device particularly well-suited for non-clinical environments.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub-combinations of the various features described hereinabove as well as variations and modifications thereof, which would occur to persons skilled in the art upon reading the foregoing description.

What is claimed is:

1. A mechanically actuated needle shielding device for metered dose syringes comprising:
a hub cap configured to be mounted over a needle hub of a syringe;
a shield slide disposed coaxially with respect to the hub cap and configured to move along a longitudinal axis between a retracted position, in which a distal end of a needle is exposed, and an extended position, in which the needle is concealed;
a shield slide magnet embedded in the shield slide;
a hub cap magnet embedded in the hub cap,
wherein the shield slide is configured to retract toward the hub cap in response to an external axial force applied during injection, and to automatically return to the extended position upon release of the force under the influence of the magnetic repelling force, thereby concealing the needle after injection and reducing the risk of accidental contact or visual exposure.

2. The device of claim 1, wherein the shielding device is mechanically coupled to the needle hub via a friction fit or threaded engagement, without requiring modification to the syringe structure.

3. The device of claim 1, wherein the shield slide is configured to retract toward the hub cap during injection to expose the needle and to automatically return to the extended position after injection under the influence of the magnetic repelling force generated between the shield slide magnet and the hub cap magnet.

4. The device of claim 1, wherein the shield slide magnet and the hub cap magnet are oriented such that like poles face one another, thereby generating a magnetic repelling force that resists axial compression of the shield slide.

5. The device of claim 1, wherein the repelling magnetic force between the shield slide magnet and the hub cap magnet stores potential energy when the shield slide is displaced toward the hub cap by an external injection force.

6. The device of claim 1, wherein the repelling magnetic force between the shield slide magnet and the hub cap magnet is configured to resist axial displacement during injection and to restore the shield slide to the extended position upon release of the injection force.

7. The device of claim 1, wherein the extended position of the shield slide results in complete enclosure of the needle to prevent accidental contact and reduce visual exposure.

8. The device of claim 1, wherein the shield slide includes a front opening aligned with the needle axis, allowing the needle to extend through the opening during injection and become enclosed after retraction of the needle.

9. The device of claim 1, wherein the hub cap includes alignment features to ensure axial and rotational stability relative to the syringe barrel during actuation of the shielding device.

10. The device of claim 1, wherein the shield slide is configured to conceal the needle prior to, during, and after injection, thereby minimizing visual exposure and reducing injection-related anxiety in pediatric patients.

11. A method of shielding a needle of a metered dose syringe using a mechanically actuated needle shielding device, the method comprising:
coupling a hub cap of the needle shielding device over a needle hub of the syringe without modifying the syringe structure;
positioning a shield slide over the needle, the shield slide being movable along a longitudinal axis between a retracted position in which the needle is exposed and an extended position in which the needle is concealed;
orienting a shield slide magnet embedded in the shield slide and a hub cap magnet embedded in the hub cap such that like poles of the magnets face one another to generate a magnetic repelling force along the longitudinal axis;
wherein during injection, the shield slide retracts toward the hub cap against the repelling magnetic force to expose the needle, and upon release of the injection force, the shield slide automatically returns to the extended position under the influence of the magnetic repelling force, whereby the needle is concealed post-injection to enhance user safety and reduce needle exposure.

12. A method of administering an injection to a pediatric patient using a syringe equipped with a mechanically actuated needle shielding device, the method comprising:
preparing the syringe with a fluid medication and a needle;
attaching a needle shielding device comprising a hub cap, a shield slide, and magnets arranged with like poles facing one another to the syringe without modifying the syringe structure;
maintaining the shield slide in an extended position to conceal the needle from the patient prior to injection;
applying an injection force, wherein the shield slide retracts along the longitudinal axis toward the hub cap to expose the needle and allow delivery of the fluid;
automatically returning the shield slide to the extended position upon release of the injection force, under the influence of the magnetic repelling force between the like-oriented magnets, whereby the needle is concealed both before and after injection, thereby reducing injection-related anxiety and enhancing post-use safety.
